(19) United States
(12) Patent Application Publication
(10) Pub. No.: US 2026/0049550 A1
(43) Pub. Date: Feb. 19, 2026

(54) EXPERIMENTAL DEVICE FOR RECONSTRUCTION AND PRODUCTION INCREASE OF NATURAL GAS HYDRATE RESERVOIRS

(71) Applicant: GUANGZHOU INSTITUTE OF ENERGY CONVERSION, CHINESE ACADEMY OF SCIENCES, Guangzhou (CN)

(72) Inventors: Xiaosen LI, Guangzhou (CN); Yi WANG, Guangzhou (CN); Jianwu LIU, Guangzhou (CN); Longhai ZHANG, Guangzhou (CN); Zhaoyang CHEN, Guangzhou (CN); Gang LI, Guangzhou (CN); Yu ZHANG, Guangzhou (CN)

(73) Assignee: GUANGZHOU INSTITUTE OF ENERGY CONVERSION, CHINESE ACADEMY OF SCIENCES, Guangzhou (CN)

(21) Appl. No.: 19/011,669

(22) Filed: Jan. 7, 2025

Related U.S. Application Data
(63) Continuation of application No. PCT/CN2024/114241, filed on Aug. 23, 2024.

(30) Foreign Application Priority Data
Aug. 19, 2024 (CN) 202411138218.3

(51) Int. Cl. E21B 49/00

(52) U.S. Cl. CPC E21B 49/00; E21B 2200/20

(57) ABSTRACT

An experimental device for reconstruction and production increase of natural gas hydrate reservoirs includes a simulation test kettle, and a high-pressure sand filling system, a fracture-forming loading system, an overburden pressure loading system, a gas pressurization system, a fracturing system and a data collection system which are respectively connected with the simulation test kettle. A reservoir medium is arranged in the simulation test kettle, the generation of fractures with various fracture widths and/or fracture shapes is simulated in the simulation reaction kettle by the fracture-forming loading system, the reservoir medium is compacted by the high-pressure sand filling system, seabed overburden pressure is simulated for the reservoir medium by the overburden pressure loading system, gas is pressurized and then enters the simulation test kettle, a fracturing fluid or proppant is injected into the simulation reaction kettle by the fracturing system, and experimental data is collected and obtained.

### CROSS REFERENCE TO THE RELATED APPLICATIONS

This application is a continuation application of International Application No. PCT/CN2024/114241, filed on Aug. 23, 2024, which is based upon and claims priority to Chinese Patent Application No. 202411138218.3, filed on Aug. 19, 2024, the entire contents of which are incorporated herein by reference.

### TECHNICAL FIELD

The present invention relates to the technical field of exploitation of natural gas hydrates, in particular to an experimental device for reconstruction and production increase of natural gas hydrate reservoirs.

### BACKGROUND

Natural gas hydrates, commonly known as combustible ice, are a type of caged ice-like crystal formed by light hydrocarbons, CO₂ and other small molecular gases and water at the low-temperature and high-pressure environment, and are widely distributed in frozen soil stratums and most unconsolidated seabed stratum pores. China is rich in natural gas hydrate reserves, and the related research has made rapid progress. In June 2007, China Geological Survey first discovered marine natural gas hydrates in Shenhu area, South China Sea, China. In November 2008, China Geological Survey discovered territorial natural gas hydrates in the permafrost region of Qilian Mountain in Qinghai Province. After continuous research on the marine natural gas hydrates and the territorial natural gas hydrates, China carried out the first trial exploitation of the natural gas hydrates in Muli Area of Qilian Mountain in September 2011. In 2017, China carried out the second trial exploitation of the natural gas hydrates in the Beipo Shenhu area Area of South China Sea, and successfully obtained natural gas from a 1,266-meter seabed argillaceous siltstone natural gas hydrate reservoir, with continuous and stable gas production for 60 days, and a cumulative gas production of 309,000 m³. In 2020, China took the lead in adopting a horizontal well drilling production technology, and carried out the second round of trial exploitation test of the natural gas hydrates in Shenhu area of South China Sea. After one month's trial exploitation, the total gas production was 861,200 m³, and the daily output was 28,700 m³, which was 5.57 times that of the first vertical well production experiment. Experiments show that argillaceous siltstone oil and gas have better development potential. However, the average daily output is still below a critical value of commercial exploitation. Wu Nengyou et al. think that the gas production required for commercial exploitation of the marine natural gas hydrates is about 5.0*10⁵ m³, which still has a gap of 2-3 orders of magnitude from the gas production of the second trial exploitation in South China Sea.

Researches show that the factors leading to low productivity are mainly embodied in the following three aspects:
Firstly, the argillaceous siltstone hydrate reservoir has extremely low permeability, which leads to extremely poor heat and mass transfer efficiency of the reservoir, and gases produced by in-situ decomposition of the hydrates are difficult to release and transport;
Secondly, in the process of depressurization exploitation, the endothermic effect accompanied by the decomposition of the hydrates often leads to the formation of secondary hydrate ices, which further block gas seepage channels; and
Thirdly, with the decomposition of the hydrates, the continuous decrease of a reservoir strength greatly increases the inducing risk of seabed disasters such as seabed landslide, seabed collapse and methane leakage.

A lot of scientific researches and engineering practices show that reservoir reconstruction is an important way to improve the production performance of unconventional low-permeability reservoirs.

Compared with the passive optimization adaptation of a primary hydrate reservoir, such as production well type design, decomposition stimulation enhancement and development strategy optimization, the reservoir reconstruction is to actively and artificially reconstruct the hydrate reservoir to improve the porosity and permeability and mechanical properties of the primary reservoir, and provide better conditions for subsequent pressure transmission and gas-water seepage exploitation processes, so as to increase the production and efficiency.

However, in the prior art, there is no device that can realize hydrate generation and exploitation and realize different reconstruction forms on the reservoir in the process.

### SUMMARY

An inventive objective of the present invention is to solve the problems existing in the prior art. The present invention provides an experimental device for reconstruction and production increase of natural gas hydrate reservoirs.

In order to solve the problems existing in the prior art, the present invention adopts the following technical solution:

An experimental device for reconstruction and production increase of natural gas hydrate reservoirs includes a simulation reaction kettle, and a high-pressure sand filling system, a fracture-forming loading system, an overburden pressure loading system, a gas pressurization system, a fracturing system and a data collection system which are respectively connected with the simulation reaction kettle, wherein
in a simulation test process, a reservoir medium is arranged in the simulation reaction kettle, the generation of fractures with various fracture widths and/or fracture shapes is simulated in the simulation reaction kettle by the fracture-forming loading system, the reservoir medium is compacted by the high-pressure sand filling system, seabed overburden pressure is simulated for the reservoir medium by the overburden pressure loading system, gas is pressurized by the gas pressurization system and then enters the simulation reaction kettle, a fracturing fluid or proppant is injected into the simulation reaction kettle by the fracturing system, and experimental data is collected and obtained by the data collection system.

As an improvement on the technical solution of the experimental device for reconstruction and production increase of natural gas hydrate reservoirs according to the present invention, at least one horizontal exploitation well and at least one vertical exploitation well are arranged in the simulation reaction kettle; and
the data collection system is capable of being arranged in the horizontal exploitation well and the vertical exploitation well to collect pressure values and/or temperature values in the horizontal exploitation well and the vertical exploitation well.

As an improvement on the technical solution of the experimental device for reconstruction and production increase of natural gas hydrate reservoirs according to the present invention, the simulation reaction kettle is provided with an end cover, a sediment loading and unloading tool and a lifting mechanism; and
the end cover seals the simulation reaction kettle, loading and unloading between sediments and the simulation reaction kettle are realized through loading and unloading of the sediments, and the lifting mechanism is used to adjust a height of the simulation reaction kettle.

As an improvement on the technical solution of the experimental device for reconstruction and production increase of natural gas hydrate reservoirs according to the present invention, a thermal insulation jacket is arranged outside the simulation reaction kettle, and the simulation reaction kettle is placed in a low-temperature water bath.

As an improvement on the technical solution of the experimental device for reconstruction and production increase of natural gas hydrate reservoirs according to the present invention, the gas pressurization system includes an air compressor, a booster pump, a gas storage tank and a pressure regulating valve which are connected in sequence, and the pressure regulating valve is communicated with the simulation reaction kettle.

As an improvement on the technical solution of the experimental device for reconstruction and production increase of natural gas hydrate reservoirs according to the present invention, the fracturing system includes a fracturing pump, an intermediate container and a stirring container which are connected in sequence, the intermediate container and the stirring container are respectively connected with the fracturing pump, and the pressure pump is communicated with the simulation reaction kettle through the intermediate container or the stirring container.

As an improvement on the technical solution of the experimental device for reconstruction and production increase of natural gas hydrate reservoirs according to the present invention, the further includes a control system, the control system includes a proportional-integral-derivative (PID) system, and the PID system is used to control exploitation pressure.

As an improvement on the technical solution of the experimental device for reconstruction and production increase of natural gas hydrate reservoirs according to the present invention, the data collection system includes a resistance measurement system, a temperature and pressure test system, a gas-liquid flow measurement and sand production measurement system, a data collection system, a computer processing system and a large centralized display screen.

As an improvement on the technical solution of the experimental device for reconstruction and production increase of natural gas hydrate reservoirs according to the present invention, the fracture-forming loading system includes a fracture-forming hydraulic cylinder, a displacement lead-out rod and a fracture-forming blade, the blade is controlled to move downwards by a hydraulic device to fracture the reservoir, and various fracture shapes and different fracture widths are capable of being simulated by changing different cutters and changing a cutter displacement.

The present invention has the beneficial effects:

According to the present invention, the reservoir pressure and temperature conditions are simulated according to a displacement mechanism and a similarity principle, the automatic measurement simulation experiment under laboratory conditions is mainly carried out with the help of the latest achievements of modern science and technology, such as computer technology and advanced sensor technology, and fracturing experiments of the hydrate reservoir, development experiments such as seepage capacity test before and after fracturing of the hydrate reservoir, hydrate formation and decomposition simulation, research on the invasion of the fracturing fluid and proppant, fracture spreading and distribution and other experiments, and simulation of the fractures with various forms and different widths can be carried out to evaluate reservoir reconstruction experiment effects.

### BRIEF DESCRIPTION OF THE DRAWINGS

Figure is a schematic structural diagram of the present invention.

Description of reference signs:

FIG. 1—simulation test kettle;

FIG. 2—high-pressure sand filling system;

FIG. 3—fracture-forming loading system;

FIG. 4—overburden pressure loading system;

FIG. 5—low-temperature water bath;

FIG. 6—thermal insulation jacket;

FIG. 7—gas pressurization system;

FIG. 8—fracturing pump;

FIG. 9—intermediate container;

FIG. 10—stirring container;

FIG. 11—air compressor;

FIG. 12—booster pump;

FIG. 13—gas storage tank;

FIG. 14—pressure regulating valve;

FIG. 15—temperature field;

FIG. 16—valve body;

FIG. 17—gas flowmeter;

FIG. 18—gas-liquid separator;

FIG. 19—electronic balance;

FIG. 20—vertical exploitation well;

FIG. 21—horizontal exploitation well;

FIG. 22—fracture-forming blade;

FIG. 23—proppant.

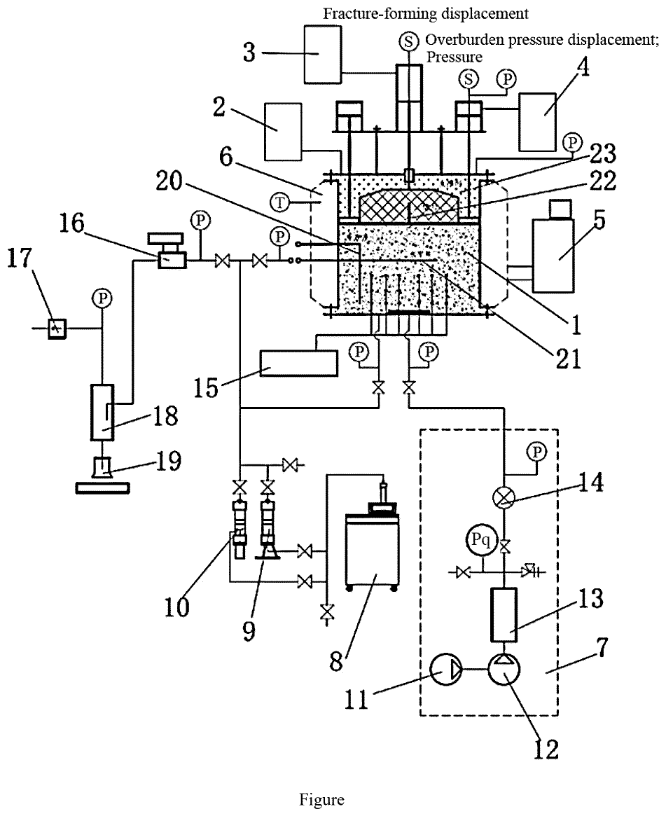

### DETAILED DESCRIPTION OF THE EMBODIMENTS

In order to clarify the inventive objectives, technical solutions and beneficial effects of the present invention, the technical solutions in embodiments of the present invention will be clearly and completely described in combination with accompanying drawings in the embodiments of the present invention. It is apparent that the described embodiments are merely part not all of the embodiments of the present invention.

An experimental device for reconstruction and production increase of natural gas hydrate reservoirs includes a simulation test kettle 1, and a high-pressure sand filling system 2, a fracture-forming loading system 3, an overburden pressure loading system 4, a gas pressurization system 7, a fracturing system and a data collection system which are respectively connected with the simulation test kettle 1.

In a simulation test process, a reservoir medium is arranged in the simulation test kettle 1, the generation of fractures with various fracture widths and/or fracture shapes is simulated in the simulation test kettle 1 by the fracture-forming loading system 3, the reservoir medium is compacted by the high-pressure sand filling system 2, and seabed overburden pressure is simulated for the reservoir medium by the overburden pressure loading system 4. Gas is pressurized by the gas pressurization system 7, and then enters the simulation test kettle 1, a fracturing fluid or proppant 23 is injected into the simulation test kettle 1 by the fracturing system, and experimental data is collected and obtained by the data collection system.

Specifically, through the verification of literature research and preliminary experiments, by means of reconstruction of injecting the fracturing fluid into the fractures of the reservoir in an exploitation process, the permeability of the reservoir can be increased under certain conditions, thereby increasing the exploitation efficiency and achieving the effect of production increase.

In the present invention, the reservoir medium is arranged in the simulation test kettle 1, and different experiments are simulated by matching with different functional systems to carry out reconstruction and production increase experiments of the natural gas hydrate reservoir.

More specifically, in the present invention, the reservoir medium is arranged in the simulation test kettle 1, the generation of fractures with various fracture widths and/or fracture shapes can be simulated in the simulation test kettle 1 through the fracture-forming loading system, the reservoir medium is compacted through the high-pressure sand filling system 2, the seabed overburden pressure is simulated for the reservoir medium through the overburden pressure loading system 4, the gas is pressurized through the gas pressurization system 7 and then enters the simulation test kettle 1, the fracturing fluid or proppant 23 is injected into the simulation test kettle 1 through the fracturing system and the experimental data is collected and obtained by the data collection system.

That is, in the present invention, since the experimental device for reconstruction and production increase of natural gas hydrate reservoirs includes the simulation test kettle 1, and the high-pressure sand filling system 2, the fracture-forming loading system 3, the overburden pressure loading system 4, the gas pressurization system 7, the fracturing system and the data collection system which are respectively connected with the simulation test kettle 1, an automatic measurement simulation experiment can be carried out in a laboratory as required. Fracturing experiments of the hydrate reservoir, development experiments such as seepage capacity test before and after fracturing of the hydrate reservoir, hydrate formation and decomposition simulation, research on the invasion of the fracturing fluid and proppant 23, fracture spreading and distribution and other experiments, and simulation of the fractures with various forms and different widths can be carried out to evaluate reservoir reconstruction experiment effects.

### Embodiment 1

During formation and decomposition of the hydrates, the reservoir medium is arranged in the simulation test kettle 1, the reservoir medium is compacted by the high-pressure sand filling system 2, and then the generation of the fractures with various fracture widths and/or fracture shapes is simulated in the simulation test kettle 1 by the fracture-forming loading system 3. In an experiment process, the seawater pressure on the reservoir medium is simulated by the overburden pressure loading system 4. Combined with gas pressurization, the pressurized gas is pressurized and then enters the simulation test kettle 1. On this basis, the development experiments such as hydrate formation and decomposition simulation can be carried out, and the simulation of the fractures with various forms and different widths can be carried out to evaluate the reservoir reconstruction experiment effects.

### Embodiment 2

Based on Embodiment 1, when it is necessary to carry out the fracturing experiments of the hydrate reservoir or the experiments of studying the invasion of the fracturing fluid and proppant 23, fracture spreading and distribution and the like, or to test the seepage capacity of the hydrate reservoir before and after fracturing, the fracturing system can be matched for use, and the fracturing fluid or proppant 23 is injected into the simulation test kettle 1 through the fracturing system, so as to achieve a fracturing effect on the reservoir medium, thereby increasing the permeability and improving the exploitation efficiency. At the same time, the simulation of the fractures with various forms and different widths can be carried out to evaluate the reservoir reconstruction experiment effects.

The simulation test kettle 1 in the present invention is used for preparing natural gas hydrate samples, the natural gas hydrate accumulation environment (temperature, pressure, sediment, gas-water-hydrate saturation) in a sedimentary layer with a water depth of 3000 meters in the South China Sea is truly simulated, and the simulation of natural gas hydrate reservoir environments under different conditions is realized under the joint action of other systems, so as to carry out different experiments.

Preferably, the simulation test kettle 1 can be a simulation test kettle 1 with a volume of 7.5L, working pressure of 40 MPa, and working temperature of −20° C. to 80° C.

In the present invention, other systems include not only the high-pressure sand filling system 2, the fracture-forming loading system 3, the overburden pressure loading system 4, the gas pressurization system 7, the fracturing system and the data collection system, but also other auxiliary systems, the other auxiliary systems including a combustible gas detection device, an explosion-proof ventilation device of a cold storage, an installation and operation platform of a test device, an instrument electrical control cabinet, etc., to ensure integrity of the present invention and improve functionality of the present invention.

In some embodiments of the present invention, the fracture-forming loading system 3 is arranged above the simulation test kettle 1, and includes a fracture-forming hydraulic cylinder, a displacement lead-out rod and a fracture-forming blade 22. The blade is controlled to move downwards by a hydraulic device to fracture the reservoir, and various fracture shapes and different fracture widths can be simulated by changing different cutters and changing a cutter displacement.

Under the joint action of the fracture-forming loading system 3 and the data collection system, a fracture-forming displacement and other data can be obtained in real time, so that experimenters obtain real-time experimental data.

In some embodiments of the present invention, the overburden pressure loading system 4 is used to simulate the seabed overburden pressure for the reservoir medium, and mainly includes a plunger pump, an overburden pressure piston and a pressure plate of the overburden pressure piston, and the overburden pressure piston is displaced by the plunger pump, so that the pressure plate of the overburden pressure piston is displaced downwards to exert the overburden pressure on the reservoir, thereby simulating the seawater pressure on the reservoir.

Under the joint action of the overburden pressure loading system 4 and the data collection system, an overburden pressure displacement, the overburden pressure and other data can be obtained in real time, so that experimenters obtain real-time experimental data.

In some embodiments of the present invention, the data collection system includes a resistance measurement system, a temperature and pressure test system, a gas-liquid flow measurement and sand production measurement system, a computer processing system and a large centralized display screen. The data collection system is used for data collection, storage, processing and display of temperature, pressure, gas-water-hydrate three-phase saturation distribution, gas-liquid injection flow, gas production, water production and sand production of the test device.

Further, the gas-liquid flow measurement and sand production measurement system includes a valve body 16, a gas-liquid separator 18 and an electronic balance 19 which are sequentially connected with the simulation test kettle 1, wherein the gas-liquid separator 18 is also connected with a gas flowmeter 17.

In some embodiments of the present invention, at least one horizontal exploitation well 21 and at least one vertical exploitation well 20 are arranged in the simulation test kettle 1. The data collection system can be arranged in the horizontal exploitation well 21 and the vertical exploitation well 20 to collect pressure values and/or temperature values in the horizontal exploitation well 21 and the vertical exploitation well 20.

In some embodiments of the present invention, not only can the reservoir medium be compacted by the high-pressure sand filling system 2, but also the fractures can be restored by the high-pressure sand filling system 2 after once experiment is completed, so as to prepare for the next experiment. Alternatively, in the current experiment, the depth, width or shape of the fracture is adjusted through filling of the high-pressure sand filling system 2.

As a specific embodiment of the present invention, one horizontal exploitation well 21 and one vertical exploitation well 20 are arranged in the simulation test kettle 1. In the horizontal exploitation well 21 and the vertical exploitation well 20, there are three layers of temperature measuring points, there are a total of 63 temperature sensors with 21 temperature sensors in each layer, and the measurement accuracy is +/−0.1° C. The pressure sensor has a measuring range of 40 MPa and an accuracy of +/−0.1%.

In some embodiments of the present invention, the simulation test kettle 1 is provided with an end cover, a sediment loading and unloading tool and a lifting mechanism. The end cover seals the simulation test kettle 1, the loading and unloading between sediments and the simulation test kettle 1 are realized through loading and unloading of the sediments, and the lifting mechanism is used to adjust a height of the simulation test kettle 1.

Further, a thermal insulation jacket 6 is arranged outside the simulation test kettle 1, and the simulation test kettle 1 is placed in a low-temperature water bath 5, so that the temperature environment of the simulation test kettle 1 during testing is ensured, and a temperature field 15 of the simulation test kettle 1 during testing is formed.

Further, quick connectors are designed on the inlet and outlet of the simulation test kettle 1 and the inlet and outlet of a piston container, and the loading and unloading are fast and convenient.

In some embodiments of the present invention, the high-pressure sand filling system 2 is arranged above the simulation test kettle 1, and mainly includes a sand filling interface and a sand storage cavity, so that the fractures can be quickly filled with sand and the proppant 23 after the fractures are formed.

In some embodiments of the present invention, the gas pressurization system 7 includes an air compressor 11, a booster pump 12, a gas storage tank 13 and a pressure regulating valve 14 which are connected in sequence, and the pressure regulating valve 14 is communicated with the simulation test kettle 1. The gas pressurization system 7 is used to pressurize the gas when the gas is quantitatively injected into the simulation test kettle 1 during hydrate formation.

In some embodiments of the present invention, the fracturing system includes a fracturing pump 8, an intermediate container 9 and a stirring container 10 which are connected in sequence, the intermediate container 9 and the stirring container 10 are respectively connected with the fracturing pump 8, and the pressure pump is communicated with the simulation test kettle 1 through the intermediate container 9 or the stirring container 10. In the present invention, the fracturing pump 8 can inject the fracturing fluid into the reservoir to achieve the fracturing effect, thereby increasing the permeability and improving the exploitation efficiency.

Under the action of the fracturing system, the fracturing effects in different strata and different fracturing modes can be simulated, the expansion of the fracturing fractures is predicted, the performance and flow law of the fracturing fluid can be analyzed, and an important basis is provided for fracturing design and optimization. Through the simulation operation, operators can understand the principle and process of a fracturing operation more deeply and master operation skills and safety specifications, thereby improving the operation efficiency and safety.

In some embodiments of the present invention, the experimental device for reconstruction and production increase of natural gas hydrate reservoirs further includes a control system, the control system includes a PID system, and the PID system is used to control exploitation pressure. With cooperation of the control system, the present invention can realize the automatic control of outlet pressure and realize the purpose of intelligent monitoring of automatic exploitation.

In some embodiments of the present invention, the data collection system includes the resistance measurement system, the temperature and pressure test system, the gas-liquid flow measurement and sand production measurement system, the data collection system, the computer processing system and the large centralized display screen.

The present invention also includes the advantages:
1, a displacement test can be carried out under simulated reservoir conditions; and the development of a plurality of related experimental technologies is met;
2, key components are imported, which can improve the reliability and accuracy of the test; the safety, accuracy and stability of key assemblies such as metering or control parts and safety protection are ensured; special materials are selected for key materials, which can not only ensure the high pressure resistance of the fluid, but also ensure the light loading and unloading of the simulation test kettle 1;
3, automatic computer collection and processing are realized, and man-machine dialogue can be realized;
4, advanced control hardware and software with reliable quality are adopted, and a reliable control method is applied to ensure stable and reliable operation of the system, so as to achieve the purpose of unattended operation;
5, the present invention adopts multiple safety protection measures, designs an electric contact pressure gauge and a safety valve, and is provided with a safety alarm system, which can set alarm values of temperature and pressure, and the instrument can give an alarm and automatically cut off the power supply under the conditions such as overvoltage and overtemperature, so as to ensure the safety of apparatuses and life; and
6, the special sediment loading and unloading tool is designed for the simulation test kettle 1, and can facilitate the loading and unloading between the sediments and the simulation kettle.

Based on the embodiments of the present invention, all other embodiments obtained by those ordinary skilled in the art without creative labor belong to the scope of protection of the present invention.

What is claimed is:

1. An experimental device for reconstruction and production increase of natural gas hydrate reservoirs, comprising a simulation reaction kettle, a high-pressure sand filling system, a fracture-forming loading system, an overburden pressure loading system, a gas pressurization system, a fracturing system, and a data collection system, wherein the high-pressure sand filling system, the fracture-forming loading system, the overburden pressure loading system, the gas pressurization system, the fracturing system, and the data collection system are respectively connected with the simulation reaction kettle;
wherein in a simulation test process, a reservoir medium is arranged in the simulation reaction kettle, generation of fractures with various fracture widths and/or different fracture shapes is simulated in the simulation reaction kettle by the fracture-forming loading system, the reservoir medium is compacted by the high-pressure sand filling system, seabed overburden pressure is simulated for the reservoir medium by the overburden pressure loading system, gas is pressurized by the gas pressurization system and then enters the simulation reaction kettle, a fracturing fluid or a proppant is injected into the simulation reaction kettle by the fracturing system, and experimental data is collected and obtained by the data collection system.

2. The experimental device according to claim 1, wherein at least one horizontal exploitation well and at least one vertical exploitation well are arranged in the simulation reaction kettle; and
the data collection system is allowed for being arranged in the at least one horizontal exploitation well and the at least one vertical exploitation well to collect pressure values and/or temperature values in the at least one horizontal exploitation well and the at least one vertical exploitation well.

3. The experimental device according to claim 1, wherein the simulation reaction kettle is provided with an end cover, a sediment loading and unloading tool, and a lifting mechanism; and
the end cover seals the simulation reaction kettle, loading and unloading between sediments and the simulation reaction kettle are realized through a loading and unloading tool of the sediments, and the lifting mechanism is used to adjust a height of the simulation reaction kettle.

4. The experimental device according to claim 3, wherein a thermal insulation jacket is arranged outside the simulation reaction kettle, and the simulation reaction kettle is placed in a low-temperature water bath.

5. The experimental device according to claim 1, wherein the gas pressurization system comprises an air compressor, a booster pump, a gas storage tank, and a pressure regulating valve, wherein the air compressor, the booster pump, the gas storage tank, and the pressure regulating valve are connected in sequence, and the pressure regulating valve is communicated with the simulation reaction kettle.

6. The experimental device according to claim 1, wherein the fracturing system comprises a fracturing pump, an intermediate container, and a stirring container, wherein the fracturing pump, the intermediate container, and the stirring container are connected in sequence, the intermediate container and the stirring container are respectively connected with the fracturing pump, and the fracturing pump is communicated with the simulation reaction kettle through the intermediate container or the stirring container.

7. The experimental device according to claim 1, further comprising a control system, wherein the control system comprises a proportional-integral-derivative (PID) system, and the PID system is used to control exploitation pressure.

8. The experimental device according to claim 1, wherein the data collection system comprises a resistance measurement system, a temperature and pressure test system, a gas-liquid flow measurement and sand production measurement system, a computer processing system, and a large centralized display screen.

9. The experimental device according to claim 1, wherein the fracture-forming loading system comprises a fracture-forming hydraulic cylinder, a displacement lead-out rod, and a fracture-forming blade, wherein the fracture-forming blade is controlled to move downwards by a hydraulic device to fracture the natural gas hydrate reservoirs, and the different fracture shapes and the various fracture widths are allowed for being simulated by changing different cutters and changing a cutter displacement.
